(19) United States
(12) Patent Application Publication
(10) Pub. No.: US 2025/0342927 A1
(43) Pub. Date: Nov. 6, 2025

(54) DETERMINING THERAPEUTIC ACTIONS BASED ON REAL-TIME SENSOR DATA CAPTURED BY RESPIRATORY DEVICES

(71) Applicant: Resper, Inc., San Francisco, CA (US)

(72) Inventors: Darren Hite, San Francisco, CA (US); James Nestor, San Francisco, CA (US); Jeremiah Robison, San Francisco, CA (US)

(21) Appl. No.: 19/196,662

(22) Filed: May 1, 2025

Related U.S. Application Data
(60) Provisional application No. 63/642,527, filed on May 3, 2024.

(51) Int. Cl. G16H 20/10; G16H 20/30; G16H 50/20; G16H 50/70

(52) U.S. Cl. CPC G16H 20/10; G16H 20/30; G16H 50/20; G16H 50/70

(57) ABSTRACT

A system for determining therapeutic actions based on real-time data captured by a respiratory device is described herein. The system receives a first set of respiratory data indicative of one or more breathing outputs from a first individual. The system applies a machine learning model to the first set of respiratory data and characteristics of the first individual to produce a therapeutic action for the first individual. The machine learning model is trained on sets of historical respiratory data, historical characteristics of individuals who produced the historical respiratory data, and historical therapeutic actions taken prior to capture of the historical respiratory data. The system causes the respiratory device to perform the therapeutic action. The system receives a second set of respiratory data from the respiratory device. The system tunes the machine learning model using the second set of respiratory data, the therapeutic action, and the characteristics of the first individual.

### CROSS REFERENCE TO RELATED APPLICATIONS

This application claims the benefit of priority to U.S. Provisional Application No. 63/642,527, filed on May 3, 2024, which is incorporated herein by reference for all purposes.

### TECHNICAL FIELD

The disclosure generally relates to the field of machine learning, and more specifically relates to using a machine learning model to perform therapeutic actions based on respiratory data.

### BACKGROUND

In current healthcare systems, treatments for individuals with respiratory conditions such as asthma, Chronic Obstructive Pulmonary Disease (COPD), or cystic fibrosis are typically prescribed on a fixed dosage schedule or on a rescue dosage basis. The fixed dosage approach involves providing patients with regular treatments (e.g., dosages of medication or therapeutic exercise), regardless of the severity or frequency of their symptoms. The rescue dosage approach is largely based on a patient's response to symptoms of their condition—for example, inhalers for asthma patients when they experience shortness of breath. Both these approaches rely on medical professionals pre-establishing a course of treatment before the onset of symptoms outside a medical setting. This can lead to treatments that sometimes might fail to fully address the patient's unique health needs because they do not account for the continuous variations in patient symptoms. Over or under addressing the patient's symptoms may yield suboptimal therapeutic results.

While many respiratory devices are capable of collecting real-time respiratory data—such as airflow, oxygen saturation, and breathing patterns—most systems lack the ability to analyze this data in real time to inform or adjust treatment dynamically. As a result, although respiratory symptoms may emerge or worsen suddenly (e.g., during an asthma attack or apnea event), current devices often fail to respond with immediate, symptom-specific therapeutic actions. Therefore, there is a pressing need for a system that can efficiently capture, process, and interpret real-time respiratory data to provide immediate, tailored treatment modifications to facilitate optimal patient care.

### SUMMARY

Systems and methods are disclosed herein for using real-time respiratory data to determine therapeutic actions to take to improve an individual's respiratory health. In some embodiments, a system receives a first set of respiratory data captured by a plurality of respiratory devices. The system receives a second set of respiratory data captured by the plurality of respiratory devices after a medication release by a respective respiratory device. The system creates training data based on a delta between the first set of respiratory data and the second set of respiratory data, an amount of medication applied, and characteristics of the individual associated with the respiratory data. The system trains a machine learning model on the training data. The system applies the machine learning model to a third set of respiratory data captured at a first respiratory device and characteristics of a first individual associated with the first respiratory device. The system receives a first amount of medication to release via the first respiratory device from the machine learning model and causes the first respiratory device to release the first amount of medication.

In some embodiments, the system receives a first set of respiratory data indicative of one or more breathing outputs from a first individual. The system applies a machine learning model to the first set of respiratory data and characteristics of the first individual to produce a therapeutic action for the first individual. The machine learning model is trained on sets of historical respiratory data captured via sensors at a plurality of respiratory devices, historical characteristics of corresponding historical individuals who produced the historical respiratory data, and historical therapeutic actions taken by the historical individuals prior to the capture of the historical respiratory data. The system causes the first respiratory device to perform the therapeutic action. The system receives a second set of respiratory data from the first respiratory device. The system tunes the machine learning model using the second set of respiratory data, the therapeutic action, and the characteristics of the first individual.

### BRIEF DESCRIPTION OF DRAWINGS

The disclosed embodiments have other advantages and features which will be more readily apparent from the detailed description, the appended claims, and the accompanying figures (or drawings). A brief introduction of the figures is below.

FIG. 1 illustrates one embodiment of a system environment for implementing a therapeutic system, in accordance with one or more embodiments.

FIG. 2 illustrates a block diagram representing training of a machine learning model, in accordance with one or more embodiments.

FIG. 3 is a diagram illustrating a computer system that implements the embodiments herein, according to one embodiment.

FIG. 4 is a flowchart for a method of causing a respiratory device to release an amount of medication, in accordance with one or more embodiments.

FIG. 5 is a flowchart for a method of causing a respiratory device to perform a therapeutic action, in accordance with one or more embodiments.

### DETAILED DESCRIPTION

The Figures (FIGS.) and the following description relate to preferred embodiments by way of illustration only. It should be noted that from the following discussion, alternative embodiments of the structures and methods disclosed herein will be readily recognized as viable alternatives that may be employed without departing from the principles of what is claimed.

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described herein.

### System Overview

Respiratory devices may be configured to measure real-time respiratory data for a user. The sensors of respiratory devices monitor several key indicators of respiratory health, such as oxygen levels, breathing rate, airflow, inhalation volume, exhalation volume, respiratory rate, end tidal carbon dioxide, heart rate, and peripheral oxygen saturation. Though respiratory devices may capture this respiratory data, conventional respiratory devices typically store the respiratory data for later analysis by a medical professional. However, these systems lack the ability to determine real-time respiratory health treatments to reduce healthcare complications or emergencies related to respiratory conditions during occurrence.

The therapeutic system described herein uses real-time respiratory data to determine immediate therapeutic actions to take to improve an individual's respiratory health. The therapeutic system uses sensor data to predict and treat possible respiratory attacks based on patterns and trends, leading to preventive care rather than reactive care. The therapeutic system may input sensor data representing one or more exhales and inhales made by an individual at a respiratory device to a machine learning model, which may output a suggestion to release medication to the user to prevent an asthma attack. For example, the therapeutic system may cause the respiratory device to release four puffs of albuterol. The therapeutic system may assess the effect of the albuterol (or another therapeutic action) on the individual based on subsequently received sensor data captured at the respiratory device and determine whether to initiate additional therapeutic actions. In another example, the therapeutic system may cause the respiratory device to lead the individual through a series of breathing exercise aimed at preventing a panic attack and use sensor data captured as the individual performs the breathing exercises to determine whether to continue with the breathing exercises, change the breathing exercises, or take another therapeutic action.

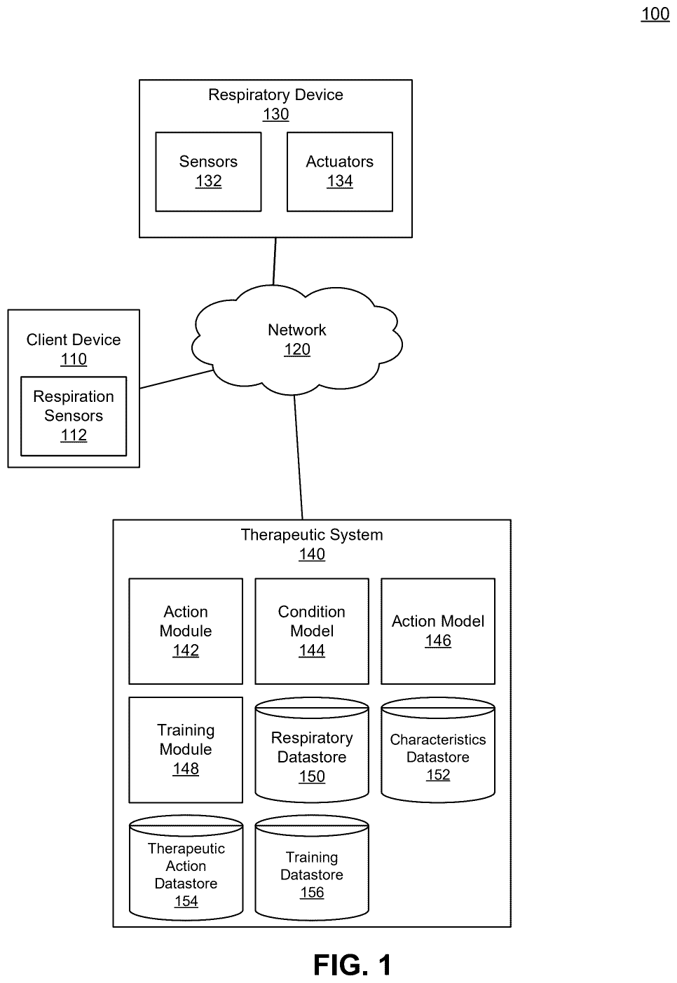

FIG. 1 illustrates one embodiment of a system environment 100 for implementing a therapeutic system 130, in accordance with one or more embodiments. As depicted in FIG. 1, the system environment includes client device 110, network 120, respiratory device 130, and therapeutic system 140. While the system environment 100 is only depicted with respect to one client device 110, this is for convenience only, and any number of client devices 110 may be interacting with therapeutic system 140. Client device 110 may be any device operated by an end-user having a user interface, such as a smartphone, a laptop, a personal computer, a wearable (e.g., smart watch), a kiosk, or any other electronic device capable of interfacing between a user and therapeutic system 140.

Therapeutic system 140 may be accessed by client device 110 using application 112. Application 112 may be an application dedicated to activities of therapeutic system 140 (e.g., an installed software package downloaded from therapeutic system 140 or an external repository such as an app store, or installed using other means such as a hard disk). Alternatively or additionally, application 112 may be a browser through which therapeutic system's 140 functionality may be accessed (e.g., directly, or indirectly through an embedded portal in a website of a third-party company).

Therapeutic system 140 communicates with other systems over the network 120. The network 120 may comprise any combination of local area and/or wide area networks, using both wired and/or wireless communication systems. In one embodiment, the network 120 uses standard communications technologies and/or protocols. For example, the network 120 includes communication links using technologies such as Ethernet, 802.11, worldwide interoperability for microwave access (WiMAX), 3G, 4G, code division multiple access (CDMA), digital subscriber line (DSL), etc. Examples of networking protocols used for communicating via the network 120 include multiprotocol label switching (MPLS), transmission control protocol/Internet protocol (TCP/IP), hypertext transport protocol (HTTP), simple mail transfer protocol (SMTP), and file transfer protocol (FTP). The network 120 may also be used to deliver push notifications through various push notification services, such as APPLE Push Notification Service (APNs) and GOOGLE Cloud Messaging (GCM). Data exchanged over the network 110 may be represented using any suitable format, such as hypertext markup language (HTML), extensible markup language (XML), or JavaScript object notation (JSON). In some embodiments, all or some of the communication links of the network 120 may be encrypted using any suitable technique or techniques.

Respiratory device 130 may be configured to detect breaths taken by a user. The respiratory device 130 may include one or more sensors 132 configured to capture respiratory data indicative of a user's respiratory health. The sensors 312 may include a pressure sensor configured to captured data indicative of pressure being applied by a user's breath and a temperature sensor configured capture data indicative of a temperature of a user's breath, among other sensors. Respiratory device 130 may also include one or more actuators 134 configured to cause a component of the respiratory device to release medication, output indica of exercises for a user to perform, and the like. The respiratory device is further described in application Ser. No. 17/382,223, filed on Jul. 21, 2021, which is incorporated by reference in its entirety.

Therapeutic system 140 determines therapeutic actions for users of respiratory devices 130 connected via network 120. Therapeutic system 140 may be instantiated on one or more servers, accessible by way of network 120. Some or all functionality of therapeutic system 140 described herein may be distributed or fully performed by application 111 on a client device 110, or vice versa. Where reference is made herein to activity performed by application 111, it equally applies that therapeutic system 140 may perform that activity off of the client device 110, and vice versa. Therapeutic system 140 includes an action module 142, condition module 144, action model 146, training module 148, respiratory datastore 150, characteristics datastore 152, therapeutic action datastore 154, and training datastore 156. In some embodiments, therapeutic system 140 includes additional or alternative components to those shown in FIG. 1.

Action module 142 determines one or more therapeutic actions to be taken for a user of a respiratory device 130. Therapeutic actions are deliberate interventions or responses performed by the device to improve, maintain, or restore a patient's respiratory function or overall respiratory health. Examples of therapeutic actions include causing a respiratory device 130 to release an amount of medication (or another substance, such as oxygen or carbon dioxide), causing the respiratory device to guide a user through a series of breathing exercises (e.g., via an audio output, a notification sent to the user's client device 110, etc.) and other breath-based interventions. Action module 142 receives sets of respiratory data from one or more respiratory devices 130 in communication with therapeutic system 140. Each set of respiratory data may describe information about a user's breathing and lung function. Respiratory data may include respiratory rate, tidal volume, minute ventilation, oxygen situation, end-tidal carbon dioxide, airflow rate, pressure levels, breath patterns, and the like. Each set of respiratory data may be associated with a time period that the respiratory data was captured by the respective respiratory device 130 and may be stored in respiratory datastore 150 in association with an identifier of the user of the respective respiratory device 130.

For received set of respiratory data, action module 142 may access characteristics associated with the user of the respective respiratory device 130 from characteristics datastore 152. The characteristics may include demographic attributes of the user (e.g., weight, age, stress levels, sleep quality, average amount of sleep per night, etc.), one or more medical conditions (e.g., asthma, anxiety, sleep apnea, altitude sickness, etc.) the user has been diagnosed with, and a medical history of the user. Each medical condition may be associated with a time of diagnosis. The medical history of the user may include a set of medical events, such as blood test results, imaging results, physical examination results, contraction of an illness, previously administered medication, and the like. Each medical event is associated with a time period during which the medical event occurred.

Action module 142 may input the characteristics associated with the user along with the received set of respiratory data to condition model 144. Condition model 144 is a machine learning model configured to identify a medical condition that the user has based on respiratory data and characteristics of the user. Condition model 144 may be trained by training module 148 on a set of condition training data stored in training datastore 156. To create the set of condition training data, training module 148 may access historical sets of respiratory data for users of respiratory devices 130 stored in respiratory datastore 150. Training module 148 may label each set of respiratory data with a medical condition with which the associated individual was diagnosed and a medical history of the associated individual. In some embodiments, training module 148 creates groups of respiratory data associated with each user together and labels the group of respiratory data with the user's medical condition(s). Training module 148 trains condition model 144 on the condition training data and stores the condition training data in training datastore 156.

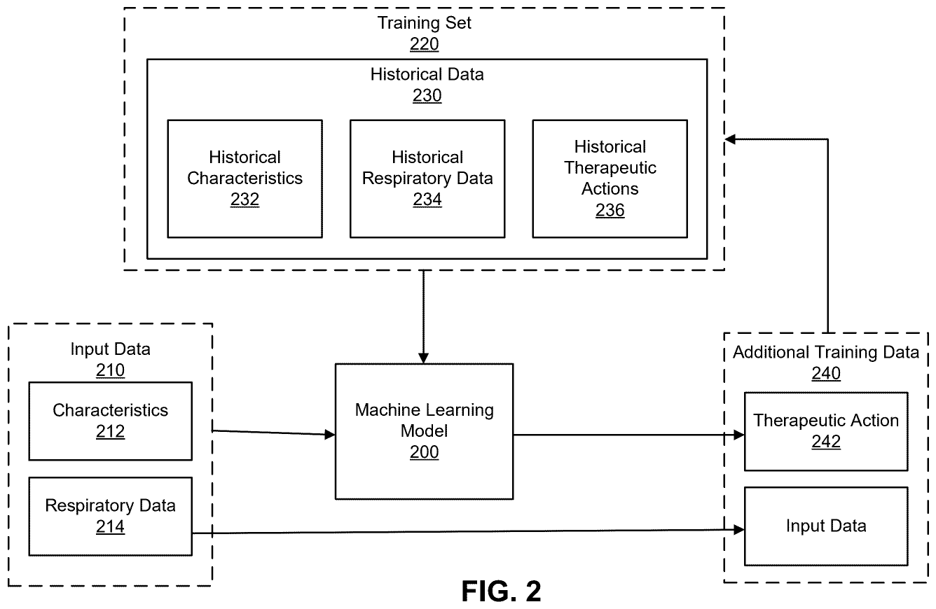

Action module 142 may input a condition determined based on a set of respiratory data with the set of respiratory data and the characteristics associated with a respective user to action model 146. In some embodiments, action module 142 also accesses previous respiratory data captured for the user from respiratory datastore 150 and inputs the previous respiratory data to action model 146 with the set of respiratory data and characteristics. In some embodiments, action module 142 does not include a medical condition determined by condition model 144 but does include one or more medical conditions formally diagnosed by a medical professional that are included in the characteristics. Action model 146 is a machine learning model configured to suggest a therapeutic action to be taken based on input respiratory data and characteristics. Action model 146 may be trained by training module 148 on action training data from training datastore 156. The action training data may include historical repository data labeled with characteristics and therapeutic actions. The action training data and training of action model 146 are further described in relation to FIG. 2.

Action module 142 may receive one or more therapeutic actions from action model 146, and action module 142 may cause the respective respiratory device 130 to perform the one or more therapeutic actions. For example, action module 142 may instruct the respective respiratory device 130 to actuate one or more mechanisms such that the respective respiratory device 130 releases a particular amount of medication or carbon dioxide. In another example, action module 142 may instruct the respective respiratory device 130 to guide the user through a series of breathing exercises by outputting audio, causing air flow in a particular direction, sending indications to a client device 110 of the user, and the like. In yet another example, action module 142 may send an alert to a medical professional, pharmacist, or other designated individual to indicate a condition the user is predicted to be experiencing based on the respiratory data. Action module 142 stores therapeutic actions along with a time period each therapeutic action was taken in therapeutic action datastore 154.

### Model Training Overview

FIG. 2 illustrates a block diagram representing training of a machine learning model 300, according to one embodiment. The following description describes training in relation to the components of FIG. 1. Alternative embodiments may include more, fewer, or different components from those illustrated in FIG. 2, and the steps may be performed in a different order from that illustrated in FIG. 2. The training may be executed by one or more processors 302 of a system, such as the therapeutic system 130. The one or more processors may include processor 302 of therapeutic system 130 executing instructions (e.g., instructions 424) that cause one or more modules to perform their respective operations.

The machine learning model 200 may be configured to select a therapeutic action for a user based on respiratory data of the user captured by a respiratory device 130. In some embodiments, the machine learning model 200 is action model 146. The training module 148 retrieves historical data 230 from local storage (e.g., respiratory datastore 150, characteristics datastore 152, and therapeutic action datastore 154). The historical data 230 may include historical characteristics 232, historical respiratory data 234, and historical therapeutic actions 236. The historical respiratory data 234 may include respiratory data captured for an associated user, where the respiratory data includes one or more sets of respiratory data each captured at an associated time period.

In some embodiments, for each set of historical respiratory data 234, the training module 148 may create a timeline of time periods that sets of respiratory data were captured and times that therapeutic actions were taken. The training module 148 may include medical events of the historical characteristics 232 in the timeline and may label the timeline with the rest of the historical characteristics (e.g., those not associated with a time or time period). Training module 148 stores the labeled timelines 232 as training data 220 (e.g., action training data).

In some embodiments, each respiratory device 130 that sent respiratory data to the therapeutic system 140 may have recorded the historical respiratory data 234 in relation to a plurality of a timestamps each representing a time the respective respiratory device 130 captured the historical respiratory data 234. The respiratory data may include a plurality of sets of historical respiratory data 234, and each set of respiratory data 234 may be associated with a respective timestamp. Each respiratory device 130 may transmit the historical respiratory data 234 to the therapeutic system 140, which stores the sets of historical respiratory data 234 in a local database in association with an identifier of a user who provided breath for the historical respiratory data 234.

Training module 148 may access the historical respiratory data 234. For each timestamp associated with a set of the historical respiratory data 234, training module 148 accesses historical characteristics 232 of a respective user that were recorded before the timestamp and one or more historic therapeutic actions that were recorded before the timestamp. Training module 148 may determine a previous set of historical respiratory data 234 captured prior to the timestamp, such that the set of historical respiratory data 234 associated the with the timestamp was captured subsequently to the previous set. Training module 148 may label the previous set with the set of historical respiratory data 234, the historical characteristics 232 recorded before the timestamp, and a historical therapeutic action 236 with a timestamp that indicates the therapeutic action was taken between times when the previous set and set of historical respiratory data 234 were captured. Training module 148 may store the labeled sets of historical respiratory data 234 as training data in training set 220 of training data.

The training data 320 includes additional training data 240 determined based on outputs from the machine learning model 300 received by the action module 142. Action module 142 receives input data 210 including respiratory data 214 captured at a respiratory device 130 and characteristics 212 of a user of the respiratory device 130. For instance, the respiratory data 214 may be recorded as the user breathes into the respiratory device 130. In some embodiments, the input data 210 also includes all or a subset of the historical data 230 associated with the user.

Action module 142 inputs the input data 210 to the machine learning model 200 and receives a therapeutic action 242 from the machine learning model 200. The action module 142 may actuate one or more components of the respiratory device 130 to cause the respiratory device 130 to perform the therapeutic action. Action module 142 may store the therapeutic action in therapeutic action datastore 154. Training module 148 may access the input data 210 and therapeutic action 242. Training module 148 may label the respiratory data 214 of the input data 210 with the characteristics 212 of the input data and the therapeutic action 242 and store the labeled respiratory data 214 as additional training data 240 in the training set 220. Training module 148 may retrain or tune the machine learning model 200 on the additional training data 200 upon receipt of the additional training data, at set intervals, upon request from an external operator, and the like.

In some embodiments, the machine-learned model 200 is a neural network that has one or more dimensions. The neural network may include different kinds of layers, such as convolutional layers, pooling layers, recurrent layers, full connected layers, and custom layers. In one embodiment, one or more custom layers may also be presented for the generation of a specific format of output. The neural network may also include nodes, kernels and/or coefficients. Training of the neural network 200 may include forward propagation and backpropagation. Each layer in a neural network may include one or more nodes, which may be fully or partially connected to other nodes in adjacent layers. In forward propagation, the neural network performs the computation in the forward direction based on outputs of a preceding layer. The operation of a node may be defined by one or more functions. The functions that define the operation of a node may include various computation operations such as convolution of data with one or more kernels, pooling, recurrent loop in RNN, various gates in LSTM, etc. The functions may also include an activation function that adjusts the weight of the output of the node. Nodes in different layers may be associated with different functions.

Each of the functions in the neural network may be associated with different coefficients (e.g. weights and kernel coefficients) that are adjustable during training. In addition, some of the nodes in a neural network may also be associated with an activation function that decides the weight of the output of the node in forward propagation. Common activation functions may include step functions, linear functions, sigmoid functions, hyperbolic tangent functions (tanh), and rectified linear unit functions (ReLU). After input is provided into the neural network and passes through a neural network in the forward direction, the results may be compared to the labels of the training data 220 to determine the neural network's performance. The process of prediction may be repeated for other inputs in the training data to compute the value of the objective function in a particular training round. In turn, the neural network performs backpropagation by using gradient descent such as stochastic gradient descent (SGD) or other optimization techniques to adjust the coefficients in various functions to improve the value of the objective function.

Multiple rounds of forward propagation and backpropagation may be performed. Training may be completed when the objective function has become sufficiently stable (e.g., the machine learning model has converged) or after a predetermined number of rounds for a particular set of training samples. The trained machine learning model can be used for performing various machine learning tasks as discussed in this disclosure.

### Computer Architecture

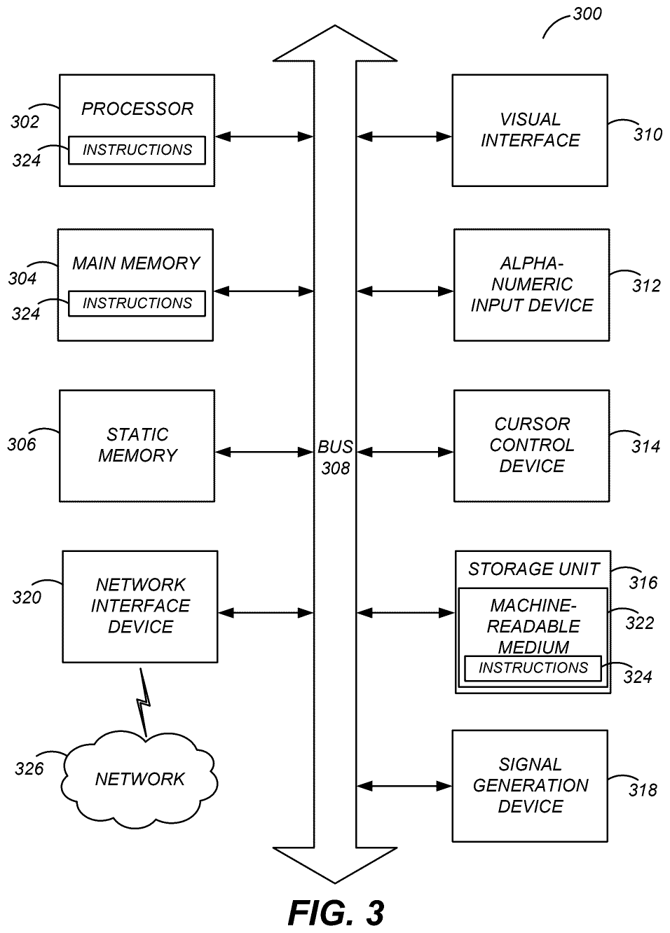

FIG. 3 is a block diagram illustrating components of an example machine able to read instructions from a machine-readable medium and execute them in a processor (or controller). Specifically, FIG. 3 shows a diagrammatic representation of a machine in the example form of a computer system 300 within which program code (e.g., software) for causing the machine to perform any one or more of the methodologies discussed herein may be executed. The program code may be comprised of instructions 324 executable by one or more processors 302. In alternative embodiments, the machine operates as a standalone device or may be connected (e.g., networked) to other machines. In a networked deployment, the machine may operate in the capacity of a server machine or a client machine in a server-client network environment, or as a peer machine in a peer-to-peer (or distributed) network environment.

The machine may be a computing system capable of executing instructions 324 (sequential or otherwise) that specify actions to be taken by that machine. Further, while only a single machine is illustrated, the term “machine” shall also be taken to include any collection of machines that individually or jointly execute instructions 324 to perform any one or more of the methodologies discussed herein.

The example computer system 400 includes one or more processors 302 (e.g., a central processing unit (CPU), a graphics processing unit (GPU), a digital signal processor (DSP), one or more application specific integrated circuits (ASICs), one or more radio-frequency integrated circuits (RFICs), field programmable gate arrays (FPGAs)), a main memory 304, and a static memory 306, which are configured to communicate with each other via a bus 308. The computer system 300 may further include visual display interface 310. The visual interface may include a software driver that enables (or provide) user interfaces to render on a screen either directly or indirectly. The visual interface 310 may interface with a touch enabled screen. The computer system 300 may also include input devices 312 (e.g., a keyboard a mouse), a cursor control device 314, a storage unit 316, a signal generation device 318 (e.g., a microphone and/or speaker), and a network interface device 320, which also are configured to communicate via the bus 308.

The storage unit 316 includes a machine-readable medium 322 (e.g., magnetic disk or solid-state memory) on which is stored instructions 324 (e.g., software) embodying any one or more of the methodologies or functions described herein. The instructions 324 (e.g., software) may also reside, completely or at least partially, within the main memory 304 or within the processor 302 (e.g., within a processor's cache memory) during execution.

### Example Methods

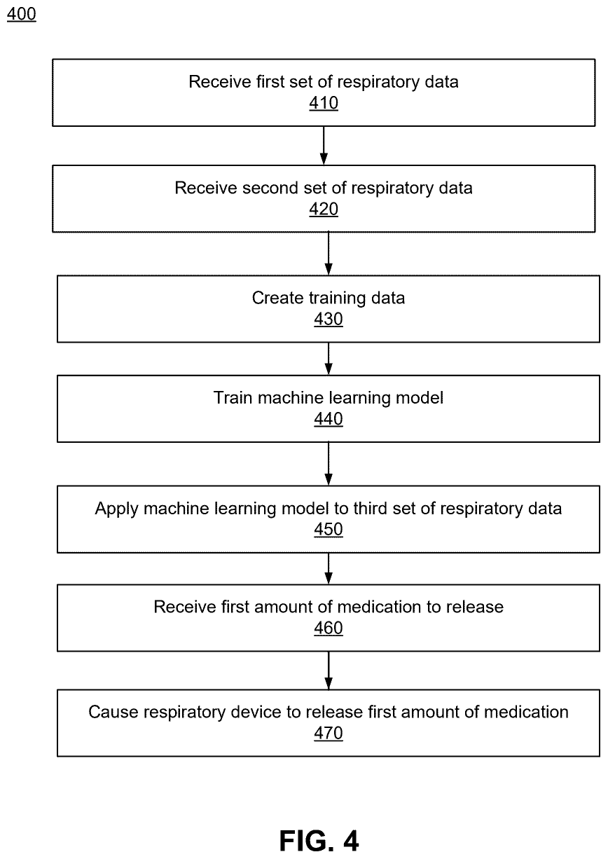

FIG. 4 is a flowchart for a method of causing a respiratory device to release an amount of medication, in accordance with one or more embodiments. Alternative embodiments may include more, fewer, or different steps from those illustrated in FIG. 4, and the steps may be performed in a different order from that illustrated in FIG. 4. Method 400 may be executed by one or more processors 302 of a system, which may include a client device 110 or respiratory device 130. The one or more processors may include processor 302 of therapeutic system 130 executing instructions (e.g., instructions 324) that cause one or more modules to perform their respective operations.

The method 400 begins with action module 142 receiving 410, from each of a plurality of respiratory devices 130, a first set of respiratory data 314 captured by one or more sensors of a respective respiratory device 130. Action module 142 receives 420 a second set of respiratory data 214 captured at each respiratory device 130 after a medication release by the respective respiratory device 130. Training module 148 creates 430 training data (e.g., action training data) based on a delta between the first set of respiratory data 214 and the second set of respiratory data 214, an amount of medication applied, and characteristics 212 of the individual associated with the respiratory data 214. In particular, training module 148 may the second set of respiratory data 214 with the difference in values from the first set of respiratory data 214, the amount of medication applied, and the characteristics 212.

Training module 148 trains 440 a machine learning model 300 (e.g., action model 146) on the training data. Action module 142 applies 450 the machine learning model 200 to a third set of respiratory data 214 captured at a first respiratory device 130 and characteristics of a first individual associated with the first respiratory device 130. Action module 142 receives 460 a first amount of medication to release via the first respiratory device 130 from the machine learning model 200. Action module 142 causes the first respiratory device 130 to release the first amount of medication. In some embodiments, action module receives a fourth set of respiratory data captured after the first amount of medication is administered and may determine a second amount of the medication to administer based on the fourth set of respiratory data. The second amount may be greater than the first amount in response to the fourth set of respiratory data indicating that a user is having more trouble breathing (e.g., rapid, shallow breathing) and may be less than the first amount in response to the fourth set of respiratory data indicating that the user's breathing is improved (e.g., slow, regulated breathing).

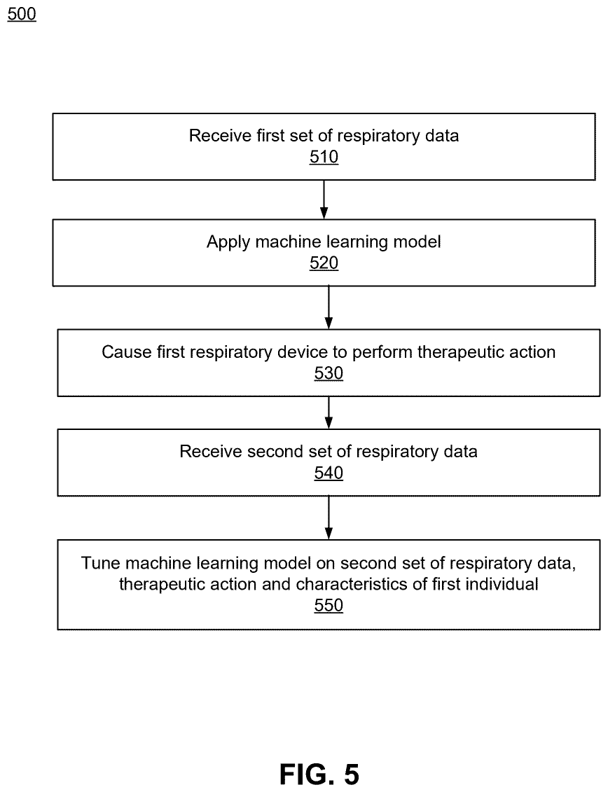

FIG. 5 is a flowchart for a method of causing a respiratory device to perform a therapeutic action, in accordance with one or more embodiments. Alternative embodiments may include more, fewer, or different steps from those illustrated in FIG. 5, and the steps may be performed in a different order from that illustrated in FIG. 5. Method 500 may be executed by one or more processors 302 of a system, which may include a client device 110 or respiratory device 130. The one or more processors may include processor 302 of therapeutic system 130 executing instructions (e.g., instructions 324) that cause one or more modules to perform their respective operations.

The method 500 begins with action module 142 receiving 510, from one or more sensors 132 of a first respiratory device 130, a first set of respiratory data 314 indicative of one or more breathing outputs from a first individual. Action module 142 applies 620 a machine learning model 200 (e.g., action model 146) to the first set of respiratory data 214 and characteristics 212 of the first individual to produce a therapeutic action 242 for the first individual. The machine learning model 200 is trained on sets of historical respiratory data 234 captured via sensors 132 at a plurality of respiratory devices 130, historical characteristics 232 of corresponding historical individuals who produced the historical respiratory data 234, and historical therapeutic actions 236 taken by the historical individuals prior to the capture of the historical respiratory data 234.

Action module 142 may determine the therapeutic action in real-time as the first individual breathes into the first respiratory device 130. Action module 142 causes 530 the first respiratory device 130 to perform the therapeutic action 242. Examples of therapeutic actions include releasing an amount of medication to improve the first individual's breathing, releasing an amount of carbon dioxide to improve the individual's carbon dioxide levels, and guiding the first individual through a set of breathing exercises to calm the first individual down from a panic attack. In some embodiments, action module 142 may send an alert to a medical professional indicative of the therapeutic action. Action module 142 receives 540 a second set of respiratory data from the first respiratory device 130. Training module 148 tunes the machine learning model 200 using the second set of respiratory data 214, the therapeutic action 242, and the characteristics 212 of the first individual.

### Alternative Embodiments

The features and advantages described in the specification are not all inclusive and in particular, many additional features and advantages will be apparent to one of ordinary skill in the art in view of the drawings, specification, and claims. Moreover, it should be noted that the language used in the specification has been principally selected for readability and instructional purposes, and may not have been selected to delineate or circumscribe the disclosed subject matter.

It is to be understood that the figures and descriptions have been simplified to illustrate elements that are relevant for a clear understanding of the present invention, while eliminating, for the purpose of clarity, many other elements found in a typical online system. Those of ordinary skill in the art may recognize that other elements and/or steps are desirable and/or required in implementing the embodiments. However, because such elements and steps are well known in the art, and because they do not facilitate a better understanding of the embodiments, a discussion of such elements and steps is not provided herein. The disclosure herein is directed to all such variations and modifications to such elements and methods known to those skilled in the art.

Some portions of above description describe the embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. It should be understood that these terms are not intended as synonyms for each other. For example, some embodiments may be described using the term “connected” to indicate that two or more elements are in direct physical or electrical contact with each other. In another example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the various embodiments. This description should be read to include one or at least one and the singular also includes the plural unless it is obvious that it is meant otherwise.

Upon reading this disclosure, those of skill in the art will appreciate still additional alternative designs for a unified communication interface providing various communication services. Thus, while particular embodiments and applications of the present disclosure have been illustrated and described, it is to be understood that the embodiments are not limited to the precise construction and components disclosed herein and that various modifications, changes and variations which will be apparent to those skilled in the art may be made in the arrangement, operation and details of the method and apparatus of the present disclosure disclosed herein without departing from the spirit and scope of the disclosure as defined in the appended claims.

What is claimed:

1. A method comprising:
receiving, from each of a plurality of respiratory devices, a first set of respiratory data captured by one or more sensors of a respective respiratory device;
receiving, from each respiratory device, a second set of respiratory data captured at the respiratory device after a medication release by the respiratory device;
creating training data based on a delta between the first set of respiratory data and the second set of respiratory data, an amount of medication applied, and characteristics of an associated individual;
training a machine learning model on the training data;
applying the machine learning model to a third set of respiratory data captured at a first respiratory device and characteristics of a first individual associated with the first respiratory device;
receiving, from the machine learning model, a first amount of medication to release via the first respiratory device; and
causing the first respiratory device to release the first amount of medication.

2. The method of claim 1, further comprising:
accessing a plurality of sets of respiratory data, wherein each set of respiratory data is associated with a historic individual;
creating the training data by labeling each set of respiratory data a delta between the set of respiratory data and a previous set of respiratory data, an amount of medication applied at a time between receipt of the previous set of respiratory data and the set of respiratory data, and characteristics of the historic individual, wherein the characteristics include a medical history of the historic individual, the medical history including at least one amount of a previously administered medication.

3. The method of claim 2, wherein the medical history includes medications previously administered to the historic individual.

4. The method of claim 1, further comprising:
accessing a plurality of sets of respiratory data, wherein each set of respiratory data is associated with a historic individual;
creating training data by labeling each set of respiratory data with a condition with which the respective historic individual was diagnosed;
training a second machine learning model on the training data, the second machine learning model configured to identify a condition of a target individual;
applying the second machine learning model to the third set of respiratory data captured at the first respiratory device;
receiving, from the second machine learning model, an identification of a condition indicated by the third set of respiratory data.

5. The method of claim 4, wherein each set of training data is further labeled with a historic medical history of the respective historic individual and wherein the second machine learning model is further applied to a first medical history of the first individual.

6. The method of claim 1, wherein the medication includes carbon dioxide.

7. The method of claim 1, wherein the characteristics include one or more of the associated individual's age, weight, height, stress levels, sleep quality, and average amount of sleep per night.

8. A method comprising:
receiving, from one or more sensors of a first respiratory device, a first set of respiratory data indicative of one or more breathing outputs from a first individual;
applying the machine learning model to the first set of respiratory data and characteristics of the first individual to produce a therapeutic action for the first individual, wherein the machine learning model is trained on 1) sets of historic respiratory data captured via sensors at a plurality of respiratory devices, 2) characteristics of corresponding historic individuals who produced the historic respiratory data, and 3) historic therapeutics taken by the historic individuals prior to the capture of the historic respiratory data;
causing the first respiratory device to perform the therapeutic action;
receiving, from the first respiratory device, a second set of respiratory data; and
tuning the machine learning model using the second set of respiratory data, the therapeutic action, and the characteristics of the first individual.

9. The method of claim 8, wherein the therapeutic action is release of an amount of medication at the first respiratory device.

10. The method of claim 8, wherein the therapeutic action is guidance of the first individual through a set of breathing exercises.

11. The method of claim 8, further comprising:
sending an alert to a medical professional indicative of the therapeutic action.

12. The method of claim 8, the method further comprising:
inputting, to a second machine learning model, one or more sets of respiratory data captured at the first respiratory device, one or more therapeutic actions performed by the first respiratory device, and characteristics of the first individual; and
receiving, from the second machine learning model, the identified condition of the first individual;
wherein the input to the machine learning model includes an identified condition of the first individual.

13. The method of claim 12, wherein the second machine learning model is trained on sets of respiratory data, each set of respiratory data associated with a historic individual of a plurality of historic individuals and labeled with one or more therapeutic actions performed by a respective respiratory device and characteristics of the respective historic individual.

14. The method of claim 8, wherein the therapeutic action is determined in real-time as the first individual breathes into the first respiratory device.

15. A non-transitory computer-readable storage medium storing instructions that, when executed, cause a processor to perform steps comprising:
receiving, from one or more sensors of a first respiratory device, a first set of respiratory data indicative of one or more breathing outputs from a first individual;
applying the machine learning model to the first set of respiratory data and characteristics of the first individual to produce a therapeutic action for the first individual, wherein the machine learning model is trained on 1) sets of historic respiratory data captured via sensors at a plurality of respiratory devices, 2) characteristics of corresponding historic individuals who produced the historic respiratory data, and 3) historic therapeutics taken by the historic individuals prior to the capture of the historic respiratory data;
causing the first respiratory device to perform the therapeutic action;
receiving, from the first respiratory device, a second set of respiratory data; and
tuning the machine learning model using the second set of respiratory data, the therapeutic action, and the characteristics of the first individual.

16. The non-transitory computer-readable storage medium of claim 15, wherein the therapeutic action is release of an amount of medication at the first respiratory device.

17. The non-transitory computer-readable storage medium of claim 15, wherein the therapeutic action is guidance of the first individual through a set of breathing exercises.

18. The non-transitory computer-readable storage medium of claim 15, the steps further comprising:
sending an alert to a medical professional indicative of the therapeutic action.

19. The non-transitory computer-readable storage medium of claim 15, the steps further comprising:
inputting, to a second machine learning model, one or more sets of respiratory data captured at the first respiratory device, one or more therapeutic actions performed by the first respiratory device, and characteristics of the first individual; and
receiving, from the second machine learning model, the identified condition of the first individual;
wherein the input to the machine learning model includes an identified condition of the first individual.

20. The non-transitory computer-readable storage medium of claim 15, wherein the therapeutic action is determined in real-time as the first individual breathes into the first respiratory device.
